(19) United States
(12) Patent Application Publication
(10) Pub. No.: US 2009/0010825 A1
(43) Pub. Date: Jan. 8, 2009

(54) Steam Delivery Device

(75) Inventors: Franco Polti, Olgiate Comasco CO (IT); Stefano Cappi, Como (IT); Peter Damiano, Mangone CS (IT)

Correspondence Address: NOTARO & MICHALOS P.C., 100 DUTCH HILL ROAD, SUITE 110, ORANGEBURG, NY, 10962-2100, US

(73) Assignee: RIKEN, WAKO-SHI (JP)

(21) Appl. No.: 12/281,218

(22) PCT Filed: Mar. 21, 2006
(86) PCT No.: PCT/IT06/00174

§ 371 Date: Aug. 29, 2008

(51) Int. Cl. A61L 2/07

(52) U.S. Cl. 422/298; 422/292; 422/300

(57) ABSTRACT

Steam delivery device comprising a nozzle (21) from the orifice of which the steam supplied from a pressurised steam source (1) is emitted. The delivery device is provided with a tubular chamber (14) positioned around the zone where the orifice of the nozzle (21) emerges, said tubular chamber (14) being closed at its end close to the orifice of the nozzle (21) and open at the opposite end (14b) through which the steam is delivered into the environment towards the objects to be sterilized.

The present invention relates to a steam delivery device comprising a nozzle from the orifice of which the steam supplied from a pressurised steam source is emitted.

It is known that steam, which may be superheated, is used widely in all those applications which require thorough disinfection and sanitization of the environment such as, for example, train interiors, cinema halls, theatres, but also and in particular the furnishings, instruments and apparatus present in medical and veterinary clinics, dentists' studios, surgical centres, ambulances, operating theatres and similar environments.

In the dental environment, in particular, in order to deal with hygiene problems, normally costly autoclave sterilization and instrument washing equipment is used, said equipment, although being very effective, having a complex operating procedure and long process times.

This equipment is normally used once a day or after the treatment of patients with viral infections.

The set of furnishings, instruments and apparatus normally used are instead washed and sanitized by the surgery assistant, using alcohol-based disinfectants which are atomized by means of sprayers, and dried with paper towels.

This operation is indispensable after treating each patient and requires long operating times.

Systems using saturated or also superheated steam have been proposed, the latter being sprayed against the objects to be sterilized by means of a high-pressure delivery device.

However, these systems which use steam, while they are very effective for cleaning domestic or industrial environments, instead have major limitations when used for sterilization applications of the type indicated above.

In fact, in the known delivery devices, the jet of steam, no matter how powerful it is in terms of pressure and despite being supplied by the source at high temperatures, when it strikes the surfaces of the objects to be sterilized, has temperatures which are no longer suitable for the purpose, i.e. for the destruction of the microbial activity present on the said objects.

The object of the present invention is to provide a steam delivery device which allows the disinfection and sanitization of surgical and/or medical instruments and equipment, in particular but not exclusively those which are used in a dentist's studio, rapidly and in a very effective manner as regards the particular microbial activity which is to be destroyed.

The object is achieved with a steam delivery device in accordance with claim 1 below.

The invention will now be described in greater detail with reference to an example of practical embodiment, provided solely by way of a non-limiting example, illustrated in the accompanying drawings in which:

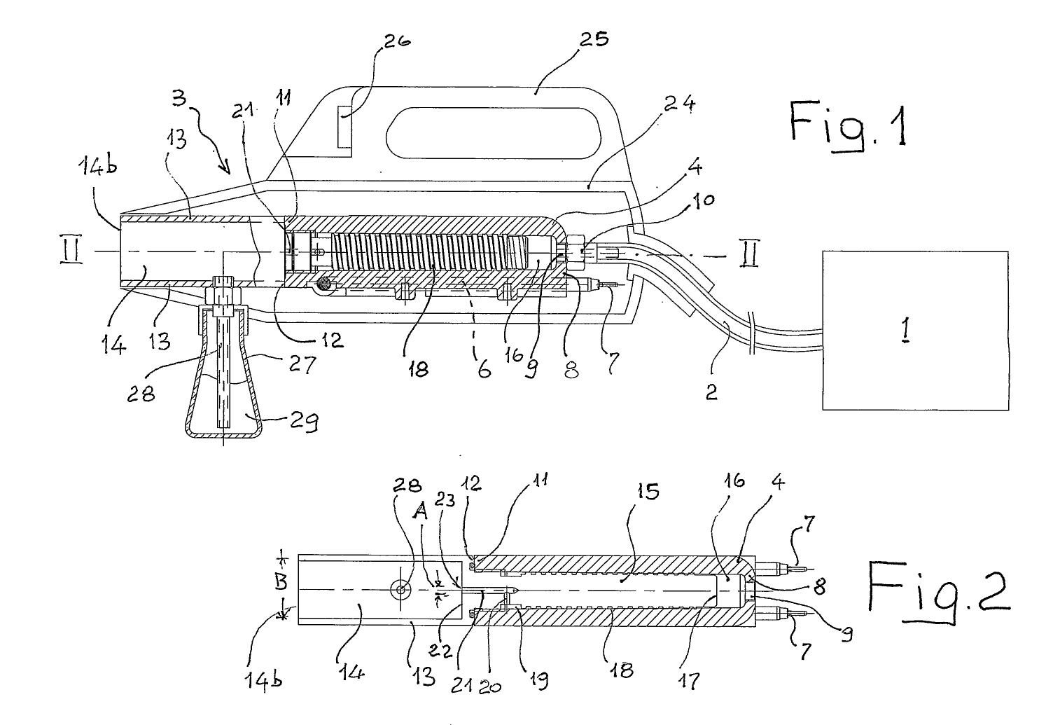

FIG. 1 shows a vertically sectioned view of the steam delivery device according to the invention;

FIG. 2 shows the horizontally sectioned view of the delivery device along the line II-II of FIG. 1.

With reference to the abovementioned figures, 1 schematically indicates a conventional steam source, the capacity of which to produce steam may be varied according to requirements. The source 1 may be, by way of example, a boiler with a 1500 W capacity supplying steam at a pressure of 3 bar or a boiler with a much higher capacity supplying steam at a pressure of 10 bar.

The steam supplied from the source 1, for example a boiler, is conveyed, by means of a pipe 2, which is for example flexible, and also conventional, to the delivery device according to the invention which is denoted overall by 3 in FIG. 1.

The latter comprises a tubular body 4 having an axial cavity 5 formed inside it. The body 4 is provided with a heating device, for example a conventional electric resistance 6 equipped with terminals 7 for the powering thereof.

At one of its ends the tubular body 4 is closed by the end wall 8 which is provided with an opening 9 to which the steam supply pipe 2 is connected by means of a union 10, shown in FIG. 1.

The opposite end 11 of the tubular body 4 is open and intended to be sealingly closed by the annular shoulder 12 of a part 13 arranged coaxially with the body 4.

This part 13 has a tubular chamber 14, preferably with a circular cross-section, which extends axially and concentrically with respect to the body 4 on one side, relative to the annular shoulder 12, and a cylindrical body 15 which extends on the opposite side, being housed inside the axial cavity 5 of the body 4 with the formation of a chamber 16 between its end 17 and the end wall 8.

The surface of the cylindrical body 15 has, formed therein, a helical groove which, with the internal cylindrical surface of the body 4, defines a helical duct 18. The latter is connected, on one side, to the chamber 16 into which the steam is supplied from the pipe 2 and, on the other side, to an annular groove 19 formed in the cylindrical body 15.

This annular groove 19 is in fluid communication with a radial duct 20 formed in the body 15 and this radial duct 20 is, in turn, in communication with a duct 21 formed axially inside the body 15, closed at the bottom end and open via an orifice 23 emerging inside the space defined by the tubular chamber 14.

The latter is therefore closed at the bottom 22 where the orifice 23 is formed and freely open with an opening 14b on the opposite side.

In accordance with the invention, the cross-sectional area “B” of the chamber 14 and its opening 14b towards the outside is greater than the cross-sectional area “A” of the orifice 23 of the duct 21 by a factor ranging between 30 and 150.

The assembly formed by the body 4 and the part 13 may be housed in a fixed location or inside a casing 24 with handle 25 for manoeuvring it, as shown in FIG. 1. The handle 25 may also have, arranged thereon, a pushbutton 26 for activating and interrupting the supply of steam from the source 1, in a conventional manner.

In accordance with the invention, the delivery device 3 may comprise a tank 27 connected radially to the part 13 and in communication with the chamber 14 by means of a pipe 28 which draws from the bottom of the tank.

The latter is intended to contain a liquid, denoted by 29 in FIG. 2, to be added to the steam during delivery thereof. The liquid is a disinfectant or a cleansing agent or an aromatic substance and suction thereof inside the chamber 14 may take place, for example, by means of the Venturi effect along the pipe 28, caused by the flow of steam being delivered.

From the above description it is clear that, following operation of the pushbutton 26 or an equivalent control device, if the apparatus is in a fixed location, the steam is supplied from the source 1 to the chamber 16 of the body 4 from where, via the helical duct 18, the groove 19 and radial duct 20, it reaches the axial duct 21 which performs the action of a delivery nozzle.

During travel along the helical duct 18, if the electric resistance 6 is activated, the steam supplied from the source 1, if required, is superheated or in any case rendered totally dry.

The steam, therefore, in the region of the orifice 23 emerges inside the space defined by the tubular chamber 14 which forms a restricted space relative to the surrounding free environment. The expansion of the steam inside the restricted environment of the chamber 14 therefore takes place with a cooling action which is notably less than that which would occur if the orifice 23 were to emerge directly inside the external free environment.

It follows that, at the outlet of the tubular section of the chamber 14, the steam still possesses a high temperature which remains substantially unvaried until it strikes the tools or the equipment to be sterilized.

The efficiency of the delivery device according to the present invention was determined by means of a series of experimental operating tests using steam supplied from a boiler at a pressure of 3.7 bar with a throughput of 100 grammes/minute, without the use of a superheater. The temperature of the steam measured at the orifice 23 was, in all the tests, kept at 100° C.

The tests were carried out detecting the temperature at which the steam, emitted from the delivery device according to the invention, managed to strike the surface to be sterilized situated at 30 mm from the opening 14b of the chamber 14 upon variation in the cross-sectional area “A” of the orifice 23 and variation in the cross-sectional area “B” of the opening 14b. The axial length of the chamber 14 was, in all the tests, equal to 120 mm.

The results of the experimental tests referred to above are shown in the following table:

### Results (Temperatures):

| | CR.SECT | | | | | | |
|---|---|---|---|---|---|---|---|
| CR.SECT | Ø 8.5 mm | Ø 13 mm | Ø 17 mm | Ø 21.5 mm | Ø 36 mm | Ø 58 mm | Free outlet |
| Ø 1.5 mm | 80° C. | 94° C. | 85° C. | 87.5° C. | 62° C. | 42° C. | 67° C. |
| Ø 2.5 mm | 83° C. | 92° C. | 90° C. | 87.5° C. | 70° C. | 49° C. | 74° C. |
| Ø 3 mm | 84° C. | 91° C. | 94° C. | 89° C. | 71° C. | 50° C. | 82° C. |

As can be seen from the data shown in the Table, the maximum efficiency, in terms of temperature still possessed by the jet of steam which leaves the delivery device according to the invention, is obtained with an orifice 23 having a diameter of 1.5 mm and with an opening 14b, of the tubular chamber 14, having a diameter of 13 mm or with an orifice having a diameter of 3 mm together with an opening 14b of the tubular chamber having a diameter of 17 mm. In both cases, the temperature of the steam measured on the surface of the object to be sterilized was equal to 94° C.

However, the efficiency in terms of temperature and therefore sterilization was acceptable also with combinations of different diameters whenever the cross-sectional area “B” of the opening 14b had a value between 30 and 150 times the cross-sectional area “A” of the orifice 23, for the same axial length of the tubular chamber 14.

The dimensions and the form of the casing 24 and the materials used for manufacture of the delivery device may obviously vary depending on the requirements, without thereby departing from the scope of the invention as described above and claimed below.

1-10. (canceled)

11: A steam delivery device comprising a nozzle from an orifice of which steam supplied from a pressurized steam source is emitted, wherein said steam delivery device is provided with a tubular chamber positioned around a zone where the orifice of the nozzle emerges, said tubular chamber being closed at a bottom end thereof and open at an opposite end (14b) thereof, through which the steam is delivered into the environment toward an object to be sterilized.

12: A device according to claim 11, wherein a cross-sectional area (B) of said tubular chamber is greater than a cross-sectional area (A) of the orifice of the nozzle.

13: A device according to claim 11, wherein a cross-sectional area (B) of said tubular chamber surrounding the zone where the orifice of the nozzle emerges is greater than a cross-sectional area (A) of the orifice of the nozzle by a factor ranging between 30 and 150.

14: A device according to claim 11, wherein a cross-section (B) of said tubular chamber is circular.

15: A device according to claim 11, wherein a said tubular chamber extends concentrically with the longitudinal axis of the nozzle over a predetermined straight section.

16: A device according to claim 11, wherein the orifice of said nozzle is situated on a closed bottom of the said tubular chamber.

17: A device according to claim 11, including a device for drying and superheating the steam, said device being for drying and super heating the steam positioned upstream of said nozzle.

18: A device according to claim 11, including a tank for containing a liquid to be added to the steam during delivery thereof, said tank being fixed to said tubular chamber and placed in communication with an interior of the tubular chamber by suction means for suctioning the liquid.

19: A device according to claim 18, wherein said liquid is one of a disinfectant and a cleansing agent and an aromatic substance.

20: A device according to claim 11, including a casing with a maneuvering handle fixed to said tubular chamber, said handle comprising a control device for activating delivery of the steam from the source toward the nozzle.
